(19) United States
(12) Patent Application Publication
(10) Pub. No.: US 2006/0242770 A1
(43) Pub. Date: Nov. 2, 2006

(54) Xyloglucan conjugates useful for modifying cellulosic textiles

(76) Inventors: Peter Albersheim, ATHENS, GA (US); Alan Darvill, Bogart, GA (US); Christian Heiss, Athens, GA (US); Warren Perkins, Vance, SC (US); Ian Hardin, Athens, GA (US); William York, Athens, GA (US)

Correspondence Address: GREENLEE WINNER AND SULLIVAN P C, 4875 PEARL EAST CIRCLE, SUITE 200, BOULDER, CO, 80301, US

(21) Appl. No.: 10/553,896

(22) PCT Filed: Apr. 16, 2004
(86) PCT No.: PCT/US04/11797

§ 371 Date: Mar. 6, 2006

Related U.S. Application Data
(60) Provisional application No. 60/464,481, filed on Apr. 21, 2003.

(51) Int. Cl. D06P 3/04; C12P 19/04

(52) U.S. Cl. 8/404; 435/101; 534/701

(57) ABSTRACT

The present invention provides xyloglucan conjugates useful as molecular anchors for attaching various functional chemical groups to cellulose or cellulosic materials. The functional groups in the xyloglucan conjugates can serve as dyes, fabric softeners, antimicrobial agents, and flame retardants and the like. Also provided are methods of preparing and using the xyloglucan conjugates of the invention.

### FIELD OF THE INVENTION

The textile industry is the primary beneficiary of the technological invention described in this patent application. The invention relates to the use of xyloglucan conjugates as molecular anchors for attaching functional chemical groups to cellulose, in particular, the cellulose fibers contained in textiles.

### BACKGROUND OF THE INVENTION

Dyes used in the textile industry are classified according to the way they are applied to the fiber. The Color Index (C.I.) lists 19 different dye classes known as “application ranges.” Of the 19, only 5 are of significance for the dyeing of cellulosic fibers [Waring, D. R. (1990) “Dyes for Cellulosic Fibers” In: The Chemistry and Application of Dyes (D. R. Waring and G. Hallas, eds.) pp. 49-106, Plenum Press, New York]. These are vat, sulfur, direct, reactive, and azoic dyes.

Vat and sulfur dyes are water-insoluble colorants that are converted into an alkali-soluble (leuco) form by a reduction process. After the leuco form is absorbed to cellulose, it is reoxidized and trapped in the fiber. Vat dyes suffer from a high cost of production and application, and sulfur dyes are limited to dull hues. These dyes are therefore steadily losing commercial value.

Direct dyes are water-soluble colored compounds that are applied to the substrate fiber directly, that is, without chemical manipulation. Direct dyes rely on their affinity for cellulose (“substantivity”) through non-covalent binding. Salt (1-5 g/l NaCl) is usually added to the dye solution to improve application efficiency. Direct dyes must be sufficiently soluble in water to enable enough dye to bind to the fiber to provide the desired color intensity. Thus direct dyes usually are characterized by poor wash fastness.

Reactive dyes are colorants that contain a reactive group capable of forming a covalent bond with the hydroxyl groups of cellulose. Accordingly, these dyes exhibit excellent wash fastness. However, the dyeing process is carried out in water, which competes with cellulose in the reaction with the dye, often leading to poor fixation efficiencies. The fixation is improved by employing very high salt concentrations (50-100 g/l NaCl), but even then the loss of the unfixed dye due to hydrolysis ranges from 20 to 50%. The large amount of unfixed dye makes extensive washing of the dyed fabric necessary, leading to a large volume of waste water.

A majority of direct and reactive dyes belong to the class of azo dyes, i.e., they contain the —N═N— linkage [Stead, C. V. (1990) “Chemistry of Azo Colorants” in Colorants and Auxiliaries 1:146-195]. Azo dyes are synthesized by reacting an aromatic amine with nitrous acid to form a diazonium salt (“diazotization”) (see FIG. 1). The azo linkage is generated from the diazonium salt by coupling it with an electron-rich aromatic compound (“coupling component”), most commonly an aminonaphthol or an aminonaphthalenesulfonic acid.

The azoic dyeing process makes use of coupling components that have substantivity for cellulose. The fabric is impregnated with the coupling component and then treated with a diazo component. The resulting azo dye is highly insoluble and binds non-covalently to cellulose. The diazo components that are normally formed in a diazotization reaction are unstable compounds that have to be prepared immediately before the coupling step. This presents the obvious disadvantage that diazotization must be carried out in the dye house. This has been alleviated to some degree by producing stable derivatives (“fast salts”) that liberate the reactive diazonium salt upon dissolution in water or chemical activation [Stead, C. V. (1990) supra]. The use of azoic dyes has declined dramatically in recent years.

The reactive dyes commonly used today comprise the class with the best fastness properties. However, these dyes generally are expensive, having the poorest application efficiency of any class of dyes. Typical efficiency of fixation of reactive dyes on cotton is only 50-80%; thus, depending on the particular dye, 20-50% is wasted. In summary, the dyeing of cellulose fabrics is plagued by intrinsic problems that cannot be solved completely within the framework of conventional methods. Dyes that bind non-covalently to cellulose have to strike a balance between the opposing characteristics of solubility and substantivity, and those that bind covalently, i.e. reactive dyes, suffer from poor application yields and the need to cope with excessive amounts of waste dye, salt, and water.

Xyloglucan is a hemicellulosic polysaccharide (FIG. 2) that is a major component (20-40%) of the primary cell walls of a wide range of plants [Hayashi, T. (1989) Ann. Rev. Plant Physiol. Plant Mol. Biol. 40:139-168]. Primary cell walls encase growing cells and the cells of the succulent tissues of plants. Primary cell walls are not lignified; lignification is a characteristic of secondary cell walls, which are the characteristic cell walls of woody tissues. Most of the xyloglucan in primary cell walls is bound tightly to the surface of cellulose microfibrils via multiple hydrophobic interactions and hydrogen bonds [Valent and Albersheim (1974) Plant Physiol. 54:105-108; Whitney et al. (1995) Plant J. 8:491-504]. Strong alkali (˜4 N KOH) is required to solubilize a majority of cellulose surface-bound xyloglucan. Although xyloglucan binds to cellulose almost instantaneously in vitro, xyloglucan is highly water-soluble when it is not bound to cellulose.

Xyloglucan functions in primary cell walls as a flexible cross-link between rigid cellulose microfibrils to form a strong, dynamic network that controls cell growth and thereby is believed to control the shapes and sizes of encased cells [Hayashi, T. (1989) supra; Carpita and Gibeaut (1993) Plant J. 3:1-30; Pauly et al. (1999) Plant J. 20:629-639]. The cellulose/xyloglucan network spontaneously assembles when newly synthesized cellulose and xyloglucan come together at the outer surface of the cell membrane. This process occurs because xyloglucan is highly water-soluble yet binds tightly to the cellulose surface immediately upon contact. The interaction of xyloglucan with cellulose plays a key role in controlling the growth of plant cells because it has the requisite physical properties of high solubility in water and avid binding to cellulose.

The valuable structural properties of xyloglucan, as with any polymer, arise as a consequence of its chemical structure [Vincken et al. (1997) Plant Physiol. 114:9-12]. Xyloglucan is structurally related to cellulose in that xyloglucan has a “cellulosic” backbone, that is, the backbone is composed of 1,4-linked β-D-glucopyranosyl (Glcp) residues. Xyloglucan is highly branched, with three out of four of the Glcp residues of most xyloglucans bearing side chains attached to O-6. Each of the side chains is composed of from 1 to 3 glycosyl residues. The side chain glycosyl residue attached directly to the backbone is almost always β-D-xylopyranosyl (Xylp). In seed xyloglucans [York et al. (1993) Carbohydr. Res. 248:285-301], a terminal β-D-galactopyranosyl (Galp) residue is attached to O-2 of many of the β-D-Xylp residues. Seed xyloglucans are the focus of this invention disclosure due to their ease of extraction, chemical and physical properties, availability in large quantities, and low cost.

The side chains of xyloglucans have profound effects on their physical properties. For example, complete removal of the side chains would produce cellulose, which is completely insoluble. Removal of some of the galactosyl residues (while leaving the underlying xylosyl residues in place) increases the viscosity of the polymer, eventually leading to gel formation [Shirakawa et al., (1998) Food Hydro colloids 12:25-28]. The rheological properties of the polymer are also affected by its molecular weight. The viscosity increases and the solubility decreases as the molecular weight of the xyloglucan increases.

The galactosyl content and molecular weight of xyloglucan can be manipulated using readily available enzymes. Galactosyl residues can be removed by fungal β-D-galactosidases [Reid et al. (1988) Enzymatic modification of natural seed gums in Gums and Stabilizers for the Food Industry 4, G. O. Phillips, D. J. Wedlock and P. A. Williams, eds. p. 391, IRL Press, Oxford, England; York et al. (1993) supra]. The molecular weight can be decreased by treatment with any of several fungal β-D-endo-1,4-glucanases, which cleave the glycosidic linkages of the regularly-spaced, unbranched β-D-Glcp residues in the xyloglucan backbone (see FIG. 2) [York et al. (1993) supra; Pauly et al. (1999) Glycobiology 9:93-100]. The unbranched, 4-linked, β-D-Glcp residues are located every fourth residue of the β-D-glucan. If the endoglucanase digestion of xyloglucan is carried out to completion, oligosaccharide subunits consisting of 7 to 9 glycosyl residues are generated (the number of residues per subunit depends on the length of the of the side chains) [York et al. (1990) Carbohydr. Res. 200:9-31]. This collection of oligosaccharides is called S₁, i.e., each S₁ oligosaccharide is a single subunit with four glucosyl residues in its backbone. Larger oligosaccharides are produced when the digestion is incomplete. For example, a collection of endoglucanase-generated xyloglucan oligosaccharides with from 14 to 18 residues is called S₂. Each S₂ oligosaccharide consists of two S₁ subunits linked together by a β-1,4-D-glucopyranoside linkage.

The seeds of a number of different legumes have been shown to contain large amounts of water-soluble xyloglucan [Kooiman, P. (1961) Res. Trav. Chim. 80:849-865], which provides a huge natural resource for the preparation of the xyloglucan conjugates disclosed herein. Most of the xyloglucan used in commercial processes comes in the form of tamarind kernel powder (TKP) prepared from the dried seeds of Tamanndus indica, a tropical legume. TKP, which is widely used in the textile industry, especially in Asia, typically is composed of approximately 60% xyloglucan, [Shankaracharya, N. B. (1998) J. Food Sci. Technol. 35:193-208]. For example, TKP is commonly used as a sizing agent during textile manufacturing. Sizing agents are applied as an aqueous solution to warp yarns in order to strengthen and lubricate them, thereby increasing the efficiency of the weaving process and improving the quality of the resulting fabric.

TKP has two major advantages over starch as a sizing agent: it is cheaper and it can be applied in smaller amounts to obtain similar results [Shankaracharya, N. B. (1998) supra]. TKP is also used as a thickener to prevent the spreading of dye during fabric printing. A patent (Racciato, 1982, U.S. Pat. No. 4,324,554) has been granted for the use of TKP as a dye antimigrant. Antimigrants are water-soluble polymers that inhibit the movement of dye particles through the capillary structure of textile fabrics during the drying process, leading to uneven deposition of dye on the fabric. Antimigrants are one of the components of virtually every formulation used for dyeing cotton as well as in continuous application processes used in the manufacture of fabrics composed of polyester/cotton blends.

GB948678 discloses a process for dyeing or printing of textiles using polysaccharides to which dye molecules are covalently linked. However, the dyeing is effected by addition of non-carbohydrate resin precondensates that are polymerized by high temperature curing. This is necessary because the polysaccharides included in this disclosure do not have strong affinity for cellulose.

U.S. Pat. No. 6,225,462 discloses a composition comprising a polysaccharide conjugate wherein a protein is covalently attached to xyloglucan to anchor it to the cellulosic fabric. The described use of the composition is an additive in laundering, and is not intended as permanent modification. The attached protein is specified to have a molecular weight of at least 5000 Daltons.

Both U.S. Pat. No. 6,225,462 and EP0930334 disclose a polysaccharide conjugate as carrier for small molecules, such as fragrances or dyes, but these are only physically adsorbed and not covalently attached to the polysaccharide and thus would not be “permanently” linked to the fabric.

Due to the limitations of the conventional dyeing methods mentioned above, there is a need in the field for a new method of dyeing that is simple, more efficient, economical and environmentally safe. Towards this end, the present application discloses new methods of dyeing cellulosic material by employing xyloglucan conjugates.

### SUMMARY OF THE INVENTION

The present invention provides xyloglucan conjugates that are useful for attaching a variety of functional chemical groups to cellulosic material. The term, “cellulosic material” as used in the present invention means any material, which is wholly or partly, made of cellulose. Examples of such material include but are not limited to paper, pulp products, and cellulosic fabrics. In the context of the present invention a cellulosic fabric is any cellulose-containing fabric known in the art, such as cotton, viscose, rayon, ramie, linen, Tencel®, or mixture thereof, or mixtures of any of these fibers, or mixtures of any of these fibers together with synthetic fibers or wool such as mixtures of cotton and spandex (stretch denim), Tencel® and wool, viscose and polyester, and cotton and wool. Paper or pulp products include lignin-containing materials such as particleboard, fiberboard, and paper.

The xyloglucan conjugates of the invention are composed of oligosaccharides ranging in size up to five hundred glycosyl residues that have a functional group covalently attached to their reducing end and/or side chains. The functional groups that can be attached to the xyloglucan conjugates include, but are not limited to, dyes, fluorescent brighteners, UV absorbers, fabric softeners, water and oil repellants, antimicrobial agents, antisoiling agents, soil release agents, stain release agents, firming agents, anti-inflammatory agents, or lubricants. The xyloglucan conjugates of the invention bind spontaneously, specifically, and so avidly to cellulose that the xyloglucan serves as a molecular anchor for the chemical covalently attached to the reducing end and/or side chains of each xyloglucan oligosaccharide. Specifically exemplified herein are xyloglucan conjugates with dye molecules covering the entire color spectrum, which show wash-fastness when applied to the cotton fabric. This method of dyeing is economical, environmentally safe, and offers a large variety of colors that are durable and color fast.

The invention also provides methods of preparing a variety of xyloglucan conjugates. Typically, the glycosidic bonds of xyloglucan polymers are partially hydrolyzed (cleaved) with enzymes to generate xyloglucan oligosaccharide (XGO) fragments ranging in size up to five hundred glycosyl residues. The enzymes useful for catalyzing such hydrolysis reactions are endoglucanases, which can be readily isolated from plants or prepared by employing recombinant technology available in the art. A functional group is then covalently attached directly to the reducing end and/or side chains of the oligosaccharide fragments to yield the xyloglucan conjugates. Alternatively, fiber-reactive dyes can be directly linked to sterically accessible hydroxyl groups along the xyloglucan chain with or without prior enzymatic digestion. In this instance, the xyloglucan conjugates thus formed are subjected to a partial endoglucanase digestion to increase solubility if necessary prior to applying to the cellulosic material. The xyloglucan conjugates of the invention can also be prepared by first digesting xyloglucan polymers exhaustively with enzymes to generate oligosaccharide fragments ranging in size, from approximately two to twenty glycosyl residues, followed by covalent attachment of a functional moiety to generate a desired xyloglucan conjugate. In this case, the resulting xyloglucan conjugates are linked to larger xyloglucan fragments before applying to the cellulosic material.

The xyloglucan conjugates of the invention are useful in a variety of applications depending upon the particular functional group attached thereto. We have used as our primary example in this application the ability of xyloglucan conjugates, each containing a dye molecule useful for dyeing fabrics. Examples of the utilities of other functional groups include fluorescent brighteners, UV absorbers, fabric softeners, water and oil repellants, antimicrobial agents, antisoiling agents, soil release agents, stain release agents, firming agents, anti-inflammatory agents, or lubricants.

### BRIEF DESCRIPTION OF THE DRAWINGS

FIG. 1 is a scheme showing the synthesis of azo dyes.

FIG. 2 shows the structure of Tamarind Seed Xyloglucan. Arrows indicate glycosidic bonds that are susceptible to attack by endoglucanase and xyloglucan endotransglycosylase.

FIG. 3 shows the action of xyloglucan endotransglycosylase (XET). Two different xyloglucan substrates are distinguished by their shading. Each oligosaccharide subunit is indicated by a rectangle.

FIG. 4 is a scheme illustrating how to prepare and use xyloglucan conjugates of the invention. Xyloglucan subunit oligosaccharides are indicated by rectangles. Dye or other functional groups are indicated by asterisks.

FIG. 5 is a scheme showing the synthesis of XGO-dye conjugates. Reaction conditions: a. aniline, NaCNBH₃, 70° C., 3 h; b. diazotized sulfanilic acid, 0-5° C., 18 h.

FIG. 6 shows examples of electrolytic oxidation of XGO and amide bond formation. Reaction conditions: a. CaBr₂, CaCO₃, graphite electrodes, 4.5 V, 25° C., 3 h; b. aniline.

FIG. 7 shows condensation of XGO with pyrazolinones. Reaction conditions: a. NaOH, EtOH, 60° C., 2 h.

FIG. 8 shows the results of the size-exclusion chromatography analysis of the initial ratio of tamarind xyloglucan to S₁-dye on the XGO size distribution after the XET reaction had gone to completion.

FIG. 9 shows the product profile of a partial digestion of Tamarind xyloglucan with endoglucanase.

FIG. 10 is the size-exclusion chromatogram of a mixture of xyloglucan obtained after the endoglucanase digestion followed by two rounds of ultrafiltration.

FIG. 11 shows the size-exclusion chromatogram of xyloglucan digestion with immobilized endoglucanase.

FIG. 12 illustrates that more than one functional chemicals can be coupled using cyanuric chloride as a branching linker.

FIG. 13 illustrates two different strategies of synthesizing XGO-azo dye conjugates.

FIG. 14 is a scheme showing the synthesis of XGO-(triphenylmethine dye).

FIG. 15 is a scheme showing the synthesis of XGO-bis-dye conjugates.

FIG. 16 illustrates that the number of subunits of xyloglucan and the dyeing temperature affect the rate and the strength of binding to the cotton fabric. The length of dyeing time is 0.5 hr for the diamonds, 2 hrs for the squares, 4.5 hrs for the triangles, and 24 hrs for the circles.

FIG. 17 shows the analysis of the xyloglucan-dye content of wash liquid from a washfastness test (AATCC Test Method 61-1989-3A). The top curve indicates the profile before the application and the bottom curve is that of the wash liquid.

### DETAILED DESCRIPTION OF THE INVENTION

In general terms and phrases used herein have their art-recognized meaning, which can be found by reference to standard textbooks, journal references and contexts known to those skilled in the art.

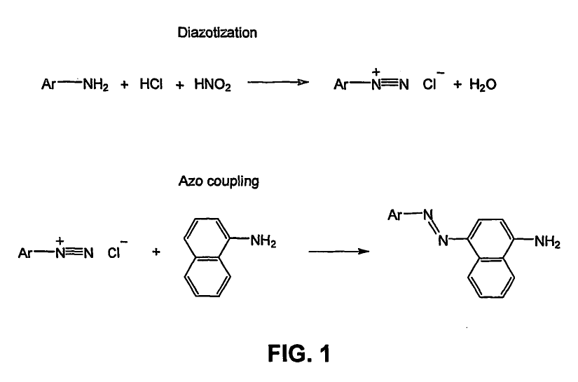

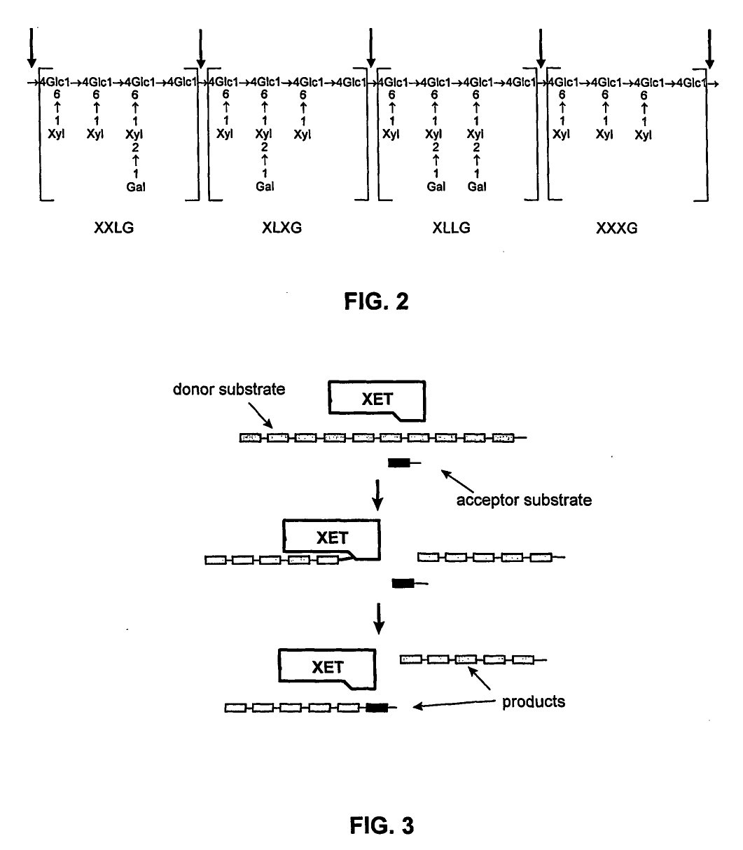

The inventors took advantage of the property of xyloglucan to bind spontaneously and avidly to the surface of cellulose to develop a new method of dyeing that alleviates problems associated with the methods that are currently used. In this application, xyloglucan serves as a molecular anchor for binding, to a cellulose-containing material, a chemical with a desired function (e.g. dye, see FIG. 1). For example, functional groups that are covalently attached to the reducing end of xyloglucan fragments rapidly and strongly adhere to the surface of cellulose-containing textiles (cotton, rayon, flax). For example, dyes that are covalently attached to xyloglucan or xyloglucan fragments are rendered highly soluble in aqueous solution, but the xyloglucan-dye conjugate binds strongly upon contact to the surface of cellulose fibers. This minimizes the loss of dye due to incomplete binding or to competing processes, such as unwanted chemical reactions, precipitation, diffusion, or binding to other surfaces. In addition to improving the efficiency of the dyeing process, this approach will reduce contamination of the environment by functional molecules (e.g. dyes) that do not bind to the fabric. Furthermore, any functional molecule, such as a dye, that is covalently attached to a xyloglucan fragment that fails to bind to the fabric can be removed from the waste stream simply by bringing it into contact with cellulose, which is an inexpensive and extremely abundant material. The xyloglucan polymers are easily obtained from inexpensive and readily available Tamarind seed meal by extraction with water [York et al. (1990) supra].

The use of the xyloglucan-conjugates of the invention is not limited to the dyeing process. Covalent modifications of the reducing end of xyloglucan fragments allow a variety of functional groups to be anchored to the surface of cellulose-containing materials. The functional groups that can be attached include molecules that soften or firm up the fabric, lubricate the fabric, make the fabric resistant to staining, endow the fabric with antimicrobial properties, or with resistance to water or oil. The following examples are provided merely for illustration purposes and do not intend to limit the scope of the invention. Compounds that can act as fabric softeners, water repellents, or lubricants when attached to xyloglucan include without limitation C₈-C₁₈ alkylamines, C₈-C₁₈ fatty acids, and siloxanes [Wagner et al. (1997) Appl. Organometal. Chem. 11:523-538]. Compounds that can act as soil releaser, stain releaser, water- and oil-repellents, and anti-soiling agents include without limitation perfluoro C₈-C₁₈ alkylamines, perfluoro C₈-C₁₈ fatty acids, and alkylanilines. Compounds that can act as UV-absorbers include 4-aminobenzoic acid and aniline derivatives. Compounds that can act as anti-microbials include dimethylhydantoin, quaternary ammonium salts, chlorhexidine, 5-chloro-2-(2,4-dichlorophenoxy)phenol, and glucoprotamine [Bohlander et al. U.S. Pat. No. 6,331,607]. Compounds that can act as fluorescent brighteners include but are not limited to stilbene derivatives, 1,2-ethylene bisbenzoxazole derivatives, 2-styrylbenzoxazole derivatives, coumarin derivatives, 1,3-diphenyl-2-pyrazoline derivatives, and naphthalamide compounds. These compounds can all be attached using triazine chemistry.

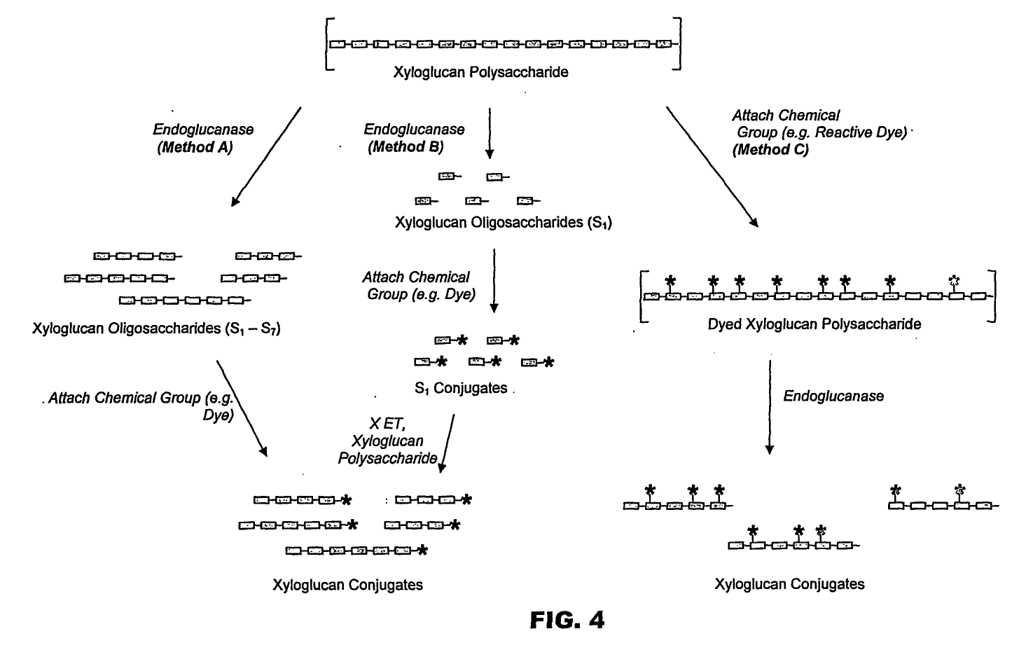

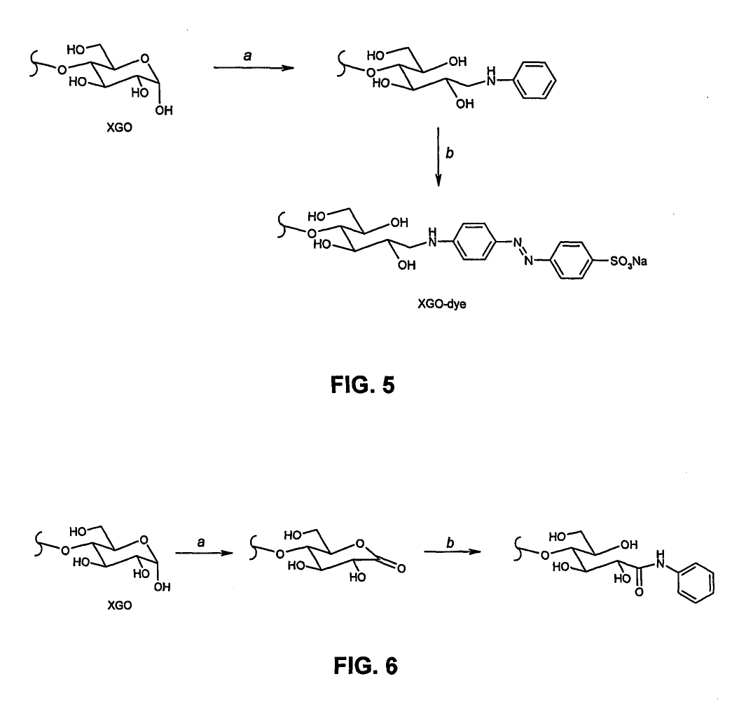

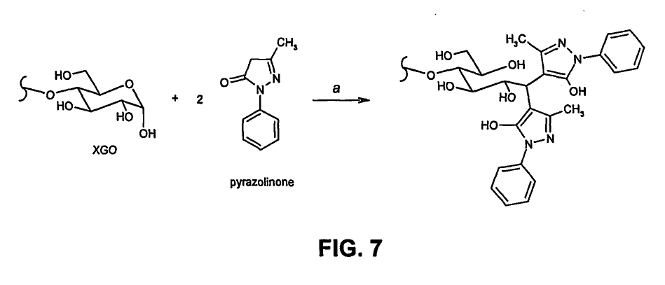

The xyloglucan conjugates of the invention are prepared by a combination of chemical and enzymatic methods. In one embodiment of the invention, xyloglucan polymer is digested partially by an endoglucanase to produce a mixture of xyloglucan oligosaccharides varying in size up to five hundred glycosyl residues (FIG. 4). The functional chemical entity (e.g. dye, see FIG. 3) is then attached chemically to the reducing ends of the collection of xyloglucan oligosaccharides (FIG. 4).

XGO-dye conjugates can also be made by attaching chromophores to the xyloglucan side chains, instead of to the reducing end. This can be done by treating xyloglucan with galactose oxidase, which converts C-6 of the galactosyl residues to an aldehyde. Dye intermediates, such as phenylenediamine, are then introduced by reductive amination and coupled to form chromophores. Alternatively, reactive dyes can be linked directly to sterically accessible hydroxyl groups in the xyloglucan side chains. Because the chromophores are randomly distributed on the polysaccharide, the ratio of chromophore to carbohydrate is independent of the length of the xyloglucan chain. This makes it unnecessary to minimize the size of the oligosaccharides to obtain an adequate chromophore content. However, the dyed xyloglucan should be small enough to be freely soluble in water. The xyloglucan can be fragmented by endoglucanase digestion either before or after the dyeing step. We have partially digested “azo-xyloglucan” (xyloglucan derivatized with Reactive Blue 19, Megazyme Cat. No. S-AZXG) and found that the resulting product binds well to cotton. In addition, we synthesized a dyed xyloglucan according to the procedure set forth in U.S. Pat. No. 4,403,032. After digestion with endoglucanase, the product was used to dye a piece of mercerized cotton. Examples 9 and 10 provide details of this approach.

Additionally, xyloglucan conjugates can be prepared by employing the following sequence: first, the xyloglucan polymer is digested completely into S₁ fragments by treatment with endoglucanase (FIG. 4). The S₁ oligosaccharides are then chemically functionalized by reaction with the appropriate chemical to give an S₁-conjugate. An enzyme called xyloglucan endotransglycosylase (XET) [Cosgrove, D. J. (1999) Ann. Rev. Plant Physiol. Mol Biol. 50:391-417] is then used to link the S₁ conjugates to xyloglucan fragments of intermediate size (two to one hundred glycosyl (sugar) residues).

XET is similar to endoglucanase in that it cleaves polymeric xyloglucan by attacking the unbranched glucosyl residues in the backbone (see FIG. 3). However, XET does not catalyze hydrolysis of the polymer. Rather, it catalyzes the formation of a new glycosidic linkage, attaching one of the fragments to the non-reducing end of another xyloglucan molecule. Therefore, XET can be used to simultaneously reduce the molecular weight of the polysaccharide and attach chemically modified xyloglucan oligosaccharides to the ends of the resulting xyloglucan fragments. An example is provided below that illustrates how XET can be used to generate xyloglucan fragments that have a dye or other surface-modifying agent attached to the reducing end.

Due to the fact that XET transfers another carbohydrate molecule instead of water, the total number of carbohydrate molecules remains constant throughout the reaction. Initially, the reaction mixture contains only very large xyloglucan (>10⁵ Da) and relatively small S₁-dye (˜10³ Da) molecules. As the reaction proceeds, the large molecules are cut and capped off with S₁-dye molecules. This process continues until equilibrium is reached where the size distribution ceases to change. The size distribution will be centered around a molecular weight that is determined by the initial mass ratio of xyloglucan to S₁-dye. For example, if that ratio is 4:1, the average molecular weight of the product will be equal to that of S₅. The polydispersity of the products will thus depend on the reaction conditions, but will generally decrease as the reaction progresses. The final size dispersion will be governed by the maximum entropy of the system.

In order to produce various chemically modified xyloglucan fragments that are small enough to have low viscosity, high solubility, and a high content of the chemical adduct, yet maintain their ability to bind to cellulose, the inventors took advantage of the recombinantly expressed enzymes such as galactosidase and endoglucanase. By judicious use of enzymes that have been cloned, over-expressed and purified, the physical properties of the xyloglucan fragments can be tailored as desired. These two enzymes have roughly opposite effects on the rheology of xyloglucan so its physical properties can be adjusted by using both enzymes in the appropriate proportion.

Tamarind xyloglucan, the raw material from which we typically derive our xyloglucan conjugates, is a large polysaccharide with a molecular weight in excess of 10⁶ Daltons. To bind efficiently to cellulose, the xyloglucan fragments should have a molecular weight between 4000 and 10000 Daltons, comprising from 3 to about 7 subunits (S₃-S₇). To obtain XGO encompassing S₃ through S₇, we initially focused our attention on a 3-step process that utilizes two enzymes. In the first step, endoglucanase is used to cleave xyloglucan into its individual subunits (S₁), secondly, a functional chemical, for instance a chromophore, is bound to the reducing glucose unit of S₁. In the final step, xyloglucan endotransglycosylase (XET), the second enzyme, reattaches the resulting S₁-conjugate to a larger piece of xyloglucan, which it obtains by cutting xyloglucan polymer.

Using this method, we prepared small quantities of a yellow and a red-violet dyes with which we dyed cotton fabric and performed qualitative wash-fastness tests. The dyes were not washed out with water, detergent, or 1 M sodium carbonate, but a portion of each dye was extracted with 1 M sodium hydroxide.

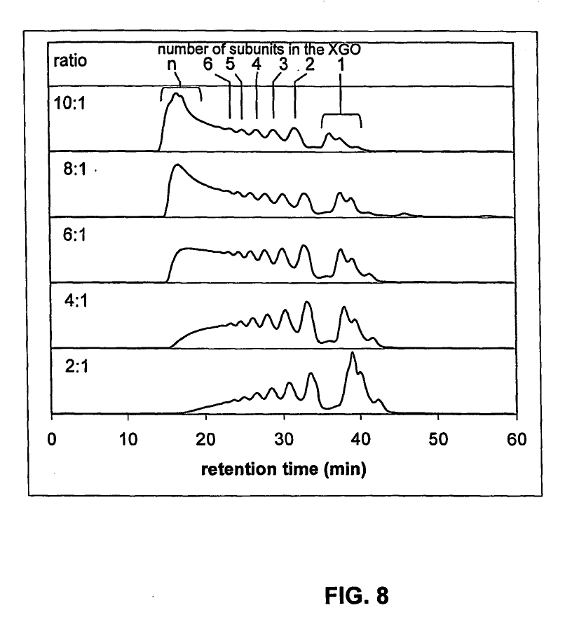

In the XET-catalyzed reaction, the sum of the molar concentrations of reactants and products is constant. Therefore, the number-average molecular weight remains the same, while the weight-average molecular weight becomes smaller. Thus, the XET reaction is a convenient way to control the molecular weight of the products simply by adjusting the stoichiometry of the reactants. In addition, we anticipated that the products of the XET reaction should have a narrow size distribution. To test this, we carried out an experiment in which we mixed an S₁-dye conjugate in varying proportions with xyloglucan polymer, and treated this mixture with XET. We analyzed the product mixture by size-exclusion chromatography (SEC), and found, as expected, that the number-average molecular weight of the product was dependent on the ratio of xyloglucan to S₁-dye and thus can be easily controlled. In addition, the size distribution narrowed as the reaction progressed. However, the product did not have as narrow a size range as we had hoped. Instead, the XET reaction reached an endpoint still containing significant amounts of polymer as well as S₁, even after extended reaction times and repeated additions of enzyme (FIG. 8).

In order to obtain the intermediate size xyloglucan fragments (S₃-S₇) we next tried incomplete endoglucanase digestion of xyloglucan. To accomplish this, it was necessary to limit the time that the xyloglucan was exposed to the endoglucanase. To this end, we attempted to remove the desired products from the mixture as soon as they were formed by carrying out the reaction in a membrane reactor. We used an Amicon® stirred-cell (Millipore, Bedford, Mass.) membrane reactor connected to a reservoir containing a xyloglucan solution. The membrane retained the enzyme as well as large xyloglucan fragments, while allowing the smaller xyloglucan fragments to pass through. Under the conditions we employed, the subunits that passed through the membrane were mostly S₁, and not intermediate size XGO (S₃S₇). Possible reasons include undersized membrane pores, insufficient membrane area, and inadequate flow rate and enzyme concentration. All the possible causes for the observed, unsatisfactory results can be addressed by using continuous, cross-flow membrane technology instead of the Amicon® stirred cell. However, if the need for pore sizes larger than 10 kDa arises, it might be necessary to modify the enzyme by increasing its molecular weight, so that it does not pass through the membrane.

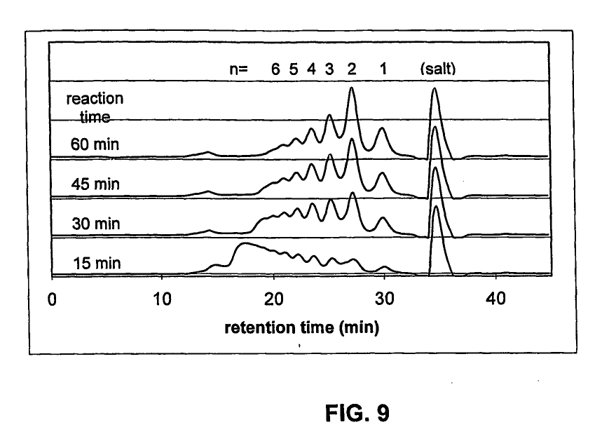

A simpler way of limiting the amount of digestion of xyloglucan by endoglucanase is by reducing the rate of the enzyme reaction. Accordingly, we cut down the amount of added enzyme and carried out the reaction at ambient temperature, which is below the optimal temperature for endoglucanase. In this way, mixtures of xyloglucan fragments with molecular weights between 1000 and 10000 Daltons could be obtained. These mixtures contained no significant amounts of large xyloglucan fragments and little S₁. However, S₂ was present in relatively high proportion (FIG. 9). Nevertheless, we used this method to routinely make gram quantities of XGO with a number-average molecular weight of ˜6500 Da.

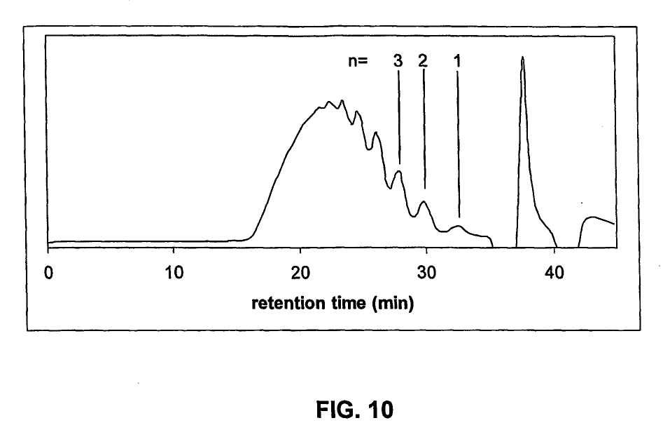

In order to avoid wasting dye molecules by attaching them to S₁ and S₂, we endeavored to remove these small molecules by ultrafiltration before the conjugation step. Prior to ultrafiltration, the enzyme has to be inactivated, which is done by raising the pH of the reaction mixture. Above pH 8, endoglucanase is, for practical purposes, inactive. FIG. 10 shows the SEC chromatogram of a mixture of XGO obtained by two ultrafiltration steps: the first filtration was through a 5-kDa membrane, keeping the retentate, which was then filtered through a 10-kDa membrane. This mixture had a number-average molecular weight of ˜30 kDa.

Seeking to be able to recover the enzyme and to optimize the size distribution of XGO, we resorted to immobilizing endoglucanase on a solid support. This should obviate the need to inactivate the enzyme, allow for reducing the amount of enzyme used, and afford greater control over the product distribution. When the endoglucanase reaction is done under homogeneous conditions, the dissolved enzyme has opportunity to cleave both native xyloglucan, as well as fragments arising from a previous turnover, resulting in high production of small oligosaccharides. When, however, the xyloglucan solution is passed through a column of immobilized enzyme, the subsequent ultrafiltration step(s) can be carried out in the absence of enzyme. By reducing the residence time on the column to only a fraction of the reaction time in the homogeneous case and feeding the retentate back into the enzyme column, the small xyloglucan fragments have less of a chance to be cut again, since they are removed from the reaction mixture shortly after being formed. By reducing the residence time progressively and increasing the number of iterations, a system of continuous removal is approached. Varying the enzyme concentration in addition to the residence time and the number of cycles should yield products of desired size.

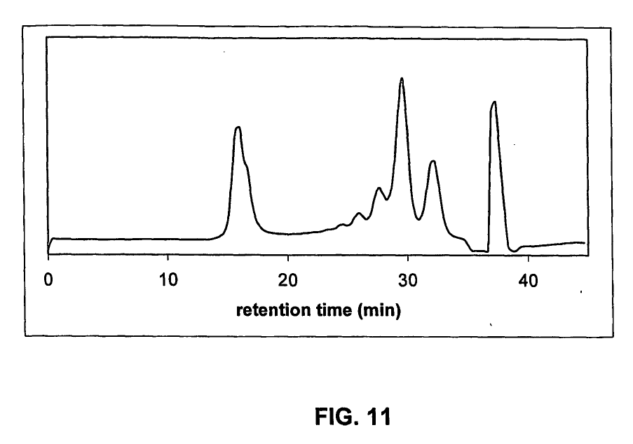

We linked endoglucanase to a commercial, cross-linked resin (AminoLink Coupling Gel, exclusion limit 5000 kDa, Pierce Chemical Company, Rockford, Ill.) and passed xyloglucan through this immobilized enzyme. Surprisingly, the product was a mixture of unchanged xyloglucan polymer and XGO fragments mostly smaller than S₃ (see FIG. 11). It appears that xyloglucan fragments arising from the first reaction cycle are able to diffuse much faster to the enzyme, which is buried largely in the pores of the solid support, than the polymer. Therefore, it would be advantageous to bind the enzyme to a non-porous solid support, where it would be located on the surface and be accessible even to large polysaccharides.

As described above, xyloglucan binds spontaneously and avidly to the surface of cellulose microfibrils. Xyloglucan's strong affinity for cellulose can be utilized in order to impart a broad range of desirable properties to cotton and other cellulosic materials. One such application is to chemically attach a dye molecule to a xyloglucan molecule to provide a novel type of dye with high water solubility and excellent substantivity for cotton. A dye molecule can be attached selectively to the reactive reducing end of a xyloglucan fragment by employing well-established chemical methods in the art, which include, but are not limited to, reductive amination [Lee et al. (1991) Carbohydr. Res. 214:155-168], oxidation followed by esterification or amide bond formation (Emmerling and Pfannemüller, 1980), or formation of a glycosylamine or aminoalditol followed by amide bond formation, or addition of carbon nucleophiles [Honda et al. (1989) Anal. Biochem. 180:351-357]. Coupling components suitable for covalent attachment to xyloglucan include but are not limited to 5-amino-1-naphthol and 7-amino-4-hydroxy-2-naphthalenesulfonic acid (J-acid). The product should contain between 3 and 10 subunits (S₃-S₁₀) to ensure its efficient binding to cellulose [Valent and Albersheim (1974) supra; Hayashi et al. (1994) Plant Cell Physiol. 35:893-899] while keeping its dye content high enough to impart intense color to the cellulose.

We used sodium cyanoborohydride in our initial efforts of reductively aminating XGO. The rate of reaction and the equilibrium concentrations of aldehyde and Schiff base are dependent of the pH of the reaction mixture. With aromatic amines, such as aniline derivatives and naphthalene derivatives, the reaction is carried out between pH 3 and pH 4 in aqueous acetate buffer.

To ensure complete conversion of XGO to the amines, we usually employ a two to ten-fold excess of amine. The excess has to be removed before proceeding to the next step in the synthesis. We discovered that amines that do not contain sulfonic acid groups can be completely removed by passing the mixture through a cation exchange resin. In contrast, amines that do feature sulfonic acid groups are not retained by cation exchangers. XGO-amine conjugates with sulfonic acid containing aromatic amines can be isolated by gel filtration. Alternatively, they can be obtained by employing S₁ instead of XGO in the reductive amination step. We carried out this reaction with H-acid (4-amino-5-hydroxy-2,7-naphthalenedisulfonic acid) and J-acid (7-amino-4-hydroxy-2-naphthalenesulfonic acid) and succeeded in separating the S₁-amine conjugates from the excess amine by reversed phase liquid chromatography. The resulting pure S₁-amine should then be suitable as substrate in an XET-catalyzed elongation.

Using the reductive amination procedure with sodium cyanoborohydride in aqueous acetate buffer, we were able to synthesize XGO conjugates from the following amines: aniline, 1,3-phenylenediamine, 1,4-phenylenediamine, 4-aminobenzylamine, 4,4′-methylenedianiline, 2,4-diaminophenol, 5-amino-1-naphthol, tris(4-aminophenyl)amine, and pararosaniline. We also synthesized S₁ conjugates with H-acid and J-acid.

The reductive amination procedure produces highest yields if the amine is sterically unencumbered. Thus, conjugates with aniline and its simple derivatives phenylenediamine and 4-aminobenzylamine were obtained in 80-90% yield, while the other, larger aniline derivatives gave conjugates in 40-50% yield. The naphthalene derivatives were conjugated in yields of 20-30%.

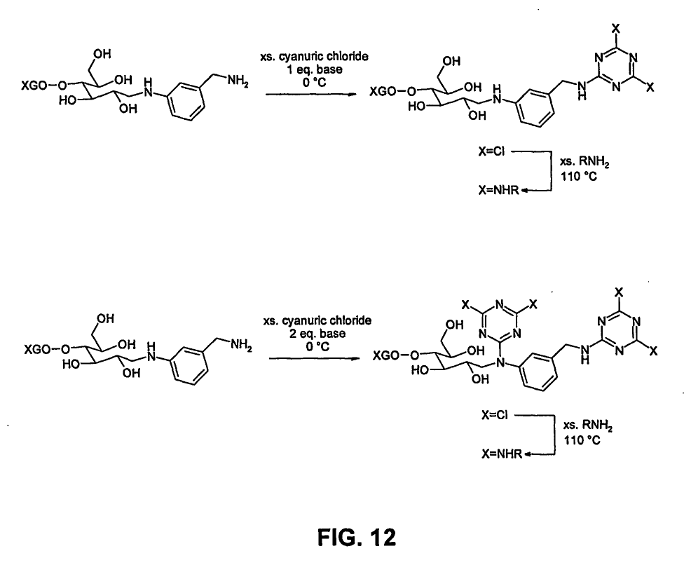

XGOs that have been derivatized at the reducing end to possess one or more sterically accessible, preferably primary, amino groups can also be furnished with multiple functional chemicals using cyanuric chloride as branching linker (FIG. 12). The functional chemicals should also have a sterically accessible amino group. Amines that can be used to incorporate sterically accessible, primary amino groups into XGO by reductive amination include ammonia, 1,2-phenylenediamine, 1,3-phenylenediamine, 1,4-phenylenediamine, 4-aminobenzylamine, and 2-(4-aminophenyl)ethylamine.

The amino-functionalized XGO is treated with an ice-cold suspension of cyanuric chloride in water, prepared according to Thurston et al. (1951), and a base, followed by an excess of the functional chemical, which also has a sterically accessible, preferably primary, amino group. As shown in FIG. 12, this method can be used to attach two (FIG. 12, top panel), four (FIG. 12, bottom panel), or more functional groups per XGO, depending on the amount of base added and on the number of available amino groups present. Six functional chemical groups can be introduced, for example, by reductively aminating XGO with an amine carrying three amino groups. Example 11 provides details of this method.

With the exception of pararosaniline, the coupled amines are not dye molecules, but rather intermediates in dye chemistry. In order to obtain XGO-dye conjugates, the amines have to undergo an azo coupling step. Two components are required for the azo coupling reaction, a diazonium salt and a coupling component. The diazonium salt is formed by treatment of an aromatic primary amine with sodium nitrite in strongly acidic solution (“diazotization”). The coupling component has to be an aromatic compound that contains at least one electron-donating group, such as amino or hydroxy.

If a compound containing two or more primary amino groups is coupled to XGO, it can be employed either as coupling component or diazonium salt. One primary amino group is converted into a secondary one during the reductive amination, forming the linkage between the aromatic amine and XGO, and is thus no longer available for diazotization. The remaining primary amino group(s), however, can be diazotized.

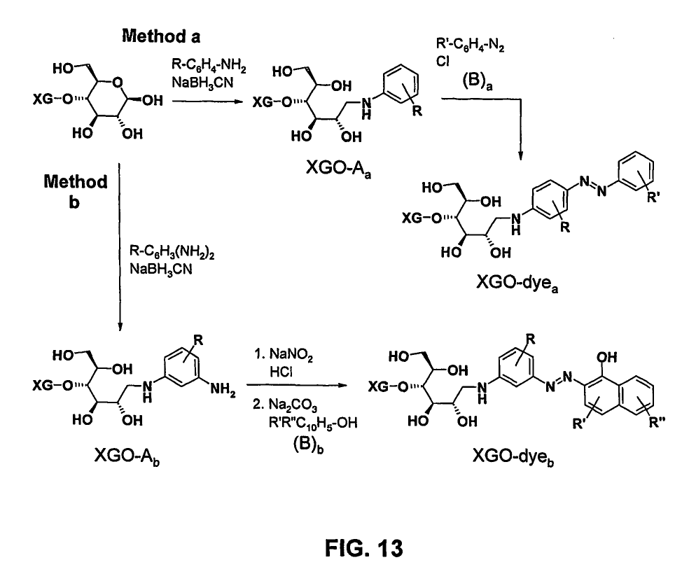

Hence, two different strategies for the azo coupling, that would lead to XGO-dye conjugates, are presented in FIG. 13: (a) azo coupling of the XGO amine with the diazonium salt of a suitable aniline, aminonaphthalene, or heteroarylamine and (b) diazotization of the remaining primary amino group(s) in the XGO amine, followed by azo coupling with any one of the numerous coupling components available to the dye chemist. While Method (a) proved to be suitable to make yellow, orange, violet, and brown XGO-dyes, red, blue, and green colors were more readily obtained by Method (b). Using these two methods, we were able to prepare xyloglucan-dye representatives over the entire color spectrum (Table 1).

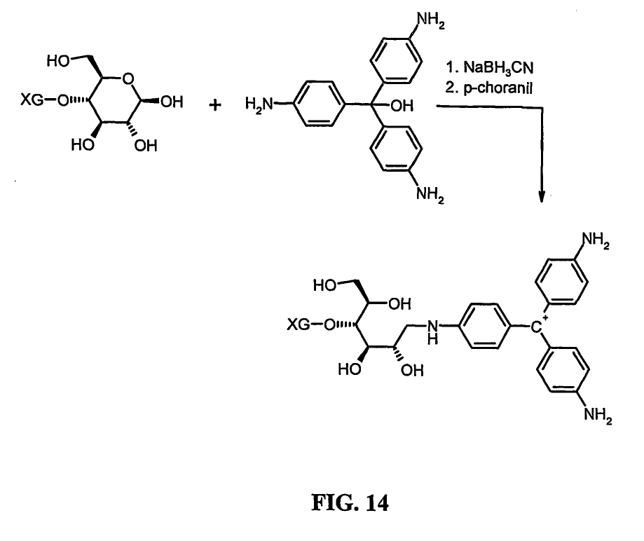

Pararosaniline, a triphenylmethine dye containing a primary amino group in the 4-position of each of its phenyl residues, was linked to XGO by reductive amination with sodium cyanoborohydride in aqueous buffer at pH 3.5. Since the dye moiety was also reduced to the leuco form, it had to be reoxidized with p-chloranil to give a violet XGO-dye (FIG. 14). Triarylmethine dyes are characterized by their bright colors and high tinctorial strengths and complement azo dyes, which usually exhibit dull colors. Closely related to the triarylmethine dyes and thus amenable for attachment to xyloglucan are also the thiazine, oxazine, and xanthene dyes.

TABLE 1
XGO-dye conjugates obtained by azo coupling
XGO-A + B −> XGO-dye

| | A | B | Method | Color (cotton) |
|---|---|---|---|---|
| 1 | aniline | Sulfanilic acid | a | yellow |
| 2 | aniline | 4-nitroaniline | a | orange |
| 3 | 1,3-phenylenediamine | 4-nitroaniline | a | brown |
| 4 | 1,3-phenylenediamine | H-acid | b | pink |
| 5 | 1,3-phenylenediamine | N-acetyl H-acid | b | pink |
| 6 | 5-amino1-naphthol | sulfanilic acid | a | red-violet |
| 7 | 1,3-phenylenediamine | 3-(4-nitrophenyl)- | b | blue |
| | | H-acid | | |
| 8 | 1,3-phenylenediamine | 3-(3-nitrophenyl)- | b | blue |
| | | H-acid | | |
| 9 | 1,3-phenylenediamine | 8-(4-nitrophenyl)- | b | red |
| | | J-acid | | |
| 10 | 1,3-phenylenediamine | 3-(4- | b | green |
| | | aminophenyl)-H- | | |
| | | acid | | |
| 11 | pararosaniline | | | violet |
| 12 | pararosaniline | 3-(3-nitrophenyl)- | b | blue |
| | | H-acid | | |
| 13 | tris(4- | 3-(3-nitrophenyl)- | b | blue |
| | aminophenyl)amine | H-acid | | |

A shortfall of the present dyeing approach lies in the fact that the number of XGO-dye molecules that can bind to cellulose is limited by the available surface area of the cellulose microfibrils. Since the major part of the XGO-dye conjugate molecule serves only to anchor the chromophore and does not contribute to the absorption of light, the final dyeing may not be sufficiently intense, even when the cellulose surface is saturated with XGO-dye molecules. Theoretically, there are three ways to address this problem: (a) the size of the xyloglucan portion of the conjugate could be reduced, (b) the extinction coefficient of the chromophore could be increased, or (c) two or more chromophores of the same kind could be attached to every XGO molecule. Approach (a) has already been addressed previously in the context of the development of the partial digestion of xyloglucan. The optimization of this enzyme reaction is expected to narrow the size distribution profile of the resulting XGO to maximize its content of S₃ and S₄. Likewise, approach (b) has been demonstrated in the preparation of XGO-dyes by azo coupling. Thus, entries 4 and 9 in Table 1 have almost identical hue in solution, yet, on cotton, entry 4 appears pink whereas entry 9 appears red. In both cases, the same number of molecules is bound to the fabric, but because entry 9, a disazo dye, has a higher extinction coefficient than entry 4, a monoazo dye, it colors the fabric more strongly.

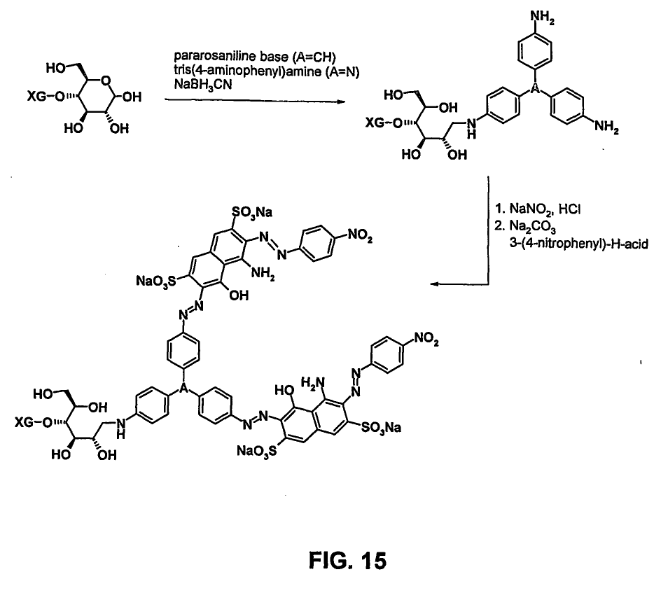

Finally, in connection with approach (c), we succeeded in making XGO-bis-dye conjugates by reductively aminating XGO with triamines, diazotizing the two remaining primary amino groups, and coupling the resulting bis-diazonium salt with a coupling component (FIG. 15). We used tris(4-aminophenyl)amine and pararosaniline as the triamines and achieved the reductive amination in yields of 40-50%. Pararosaniline was reduced to the leuco-form in the process. As coupling component we employed 3-(3-nitrophenyl)azo-H acid and obtained a green and a blue dye. The blue dye (from leuco-pararosaniline) colored cotton more deeply than its counterpart with only one chromophore (entry 8 in Table 1). The bis-dye derived from tris(4-aminophenyl)amine cannot be compared with its mono-dye counterpart because the strongly electron-donating tertiary amine imparts a significant bathochromic shift on the chromophore (601 nm→657 nm).

The number of chromophores per XGO molecule can be increased by carrying out the reductive amination with compounds containing more amino groups.

Alternatively, more chromophores could be attached by building dendritic structures onto the reducing end of XGO. Each branch of the dendrimer could then be capped off with a dye molecule.

In addition to the above process of performing an azo coupling on a derivatized oligosaccharide, preformed dye molecules can directly be linked to xyloglucan oligosaccharides. In this case, suitable dyes are not limited to azo compounds, but can include anthraquinone, phthalocyanine, and oxazine colorants, as well as stilbene-derived fluorescent brightening agents [Shore, J. (1990) “Historical Development and Classification of Colorants” In: Colorants and Auxilliarios, Vol. 1 pp. 1-31 (J. Shore, ed.) The Society of Dyers and Colorists, Bradford].

We performed dyeing experiments with the XGO-dye conjugates prepared according to the above description on a small scale in sealed test tubes. We used the unpurified dye-solutions made from 10 mg XGO-amine to dye 200 mg of mercerized cotton (cut into ˜3×3 mm pieces) at 70° C. for 8 h. After removing the dye solution and rinsing, we dried the fabric and visually inspected it to evaluate the color and its depth.

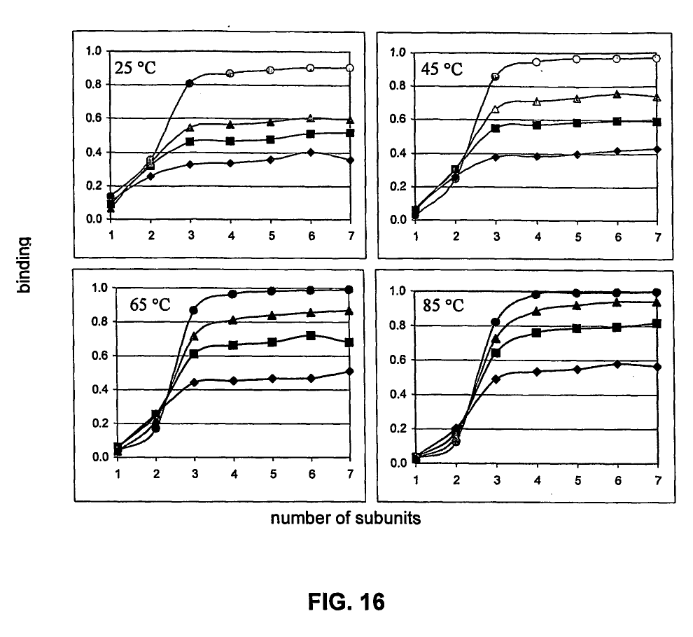

To determine the temperature dependence of the application of xyloglucan dyes, we added cotton to solutions of an XGO-dye (entry 1 in Table 1) at various temperatures and measured the amount of unbound XGO-dye at time intervals. Using SEC, we were able to follow the disappearance of the different fractions up to S₇-dye separately. Thus, we could not only determine the effect of temperature on the rate of binding, but also how the size of the XGO-dye conjugates influences the strength of binding. As might be expected, increasing the temperature accelerates the binding (FIG. 16). The size of the XGO-dye conjugates appears to matter only for the smallest fractions, S₁-, S₂-, and S₃-dye, while it does not influence the binding of larger molecules, which is nearly irreversible. S₁-dye does not bind to any appreciable extent, only about 20% of S₂-dye is attached to the fiber, and, surprisingly, the amount is reduced with increased temperature and time. S₃-dye is bound to about 80-85%, and it remains to be seen, whether this is strong enough to ensure high washfastness, and if not, it might be necessary to also remove S₃ in the process.

To obtain larger dyed cotton samples, we made a blue dye (entry 7, Table 1) from 15 g xyloglucan (the XGO-amine was not isolated) and a red dye (entry 9, Table 1) from 1.55 g XGO-3-aminophenylaminoalditol. The dye solutions were purified by ultrafiltration (MWCO=5000 Da) to remove salts and excess coupling component. Pieces of fabric from 0.01 to 0.6 m² were dyed in a batch process at 65 and 95° C.

We also tested the effect of adding salt to the dye bath. Accordingly, a salt concentration of 10 g/l markedly accelerates the dye application. Interestingly, the presence of salt did not negatively affect the uniformity of the dyeing even though the salt was added and the temperature was raised to 95° C. before submerging the cotton in the dye bath. In contrast, in conventional dyeing, salt usually has to be added gradually and the temperature raised slowly to avoid uneven application of the dye.

Wash-fastness tests were carried out according to AATCC standard procedures (AATCC Test Method 61-1989). The various conditions represent accelerated tests designed to simulate home or commercial launderings. Test No. 2A simulates 5 home or commercial launderings at 38° C., Test No. 3A simulates 5 home launderings at 60° C. or 5 commercial launderings at 49° C., and Test No. 4A simulates 5 home launderings at 63° C. with 5% available chlorine or 5 commercial launderings at 71° C. with 1% available chlorine. The rating scale goes from 1 to 5, where 5 is the best rating, indicating negligible or no color change or color transfer.

Results are summarized in Table 2.

TABLE 2
Wash-fastness ratings of different dyed fabric samples

| | | Number | | | |
|---|---|---|---|---|---|
| Entry | XGO Size | Average | Dyeing | Test | Fastness |
| Number | Distribution | MW | Temperature | Conditions | Rating |
| 1 | See FIG. 9 | 6500 | 65 | 3A | 2-3 |
| | (45 min) | | | | |
| 2 | See FIG. 9 | 6500 | 65 | 4A | 2-3 |
| | (45 min) | | | | |
| 3 | See FIG. 9 | 6500 | 95 | 2A | 4 |
| | (45 min) | | | | |
| 4 | See FIG. 9 | 6500 | 95 | 3A | 3-4 |
| | (45 min) | | | | |
| 5 | See FIG. 10 | 30000 | 95 | 3A^{a} | 4-5 |

^{a}Due to equipment failure, test conditions were not identical, therefore result for entry 5 has to be judged as preliminary

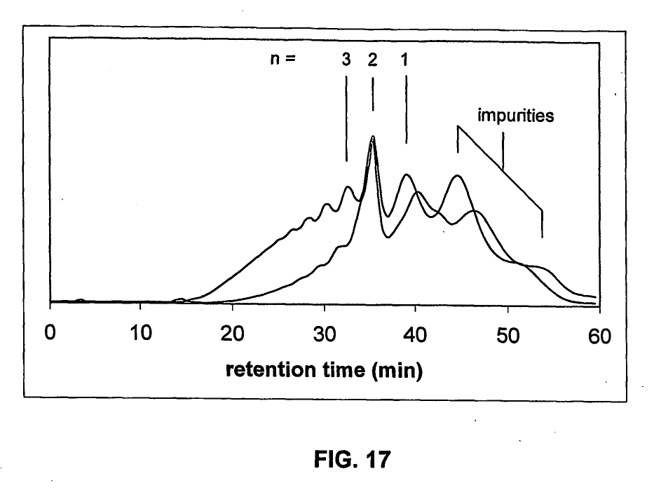

Taking into consideration that the dyes (except for entry 5) had not been purified, and therefore still contained S₁, S₂, and S₃ conjugates before the application, these fastness results are remarkable. To confirm that the S₁, S₂, and S₃ conjugates are responsible for the observed color change, we analyzed the wash liquid after the 3A test by SEC. The chromatogram (FIG. 17) allowed us to estimate that almost 60% of the washed out color came from S₁- and S₂-dye, over 30% from impurities (possibly unconjugated dye or dye-precursors, which can be avoided by using exact stoichiometry), and less than 10% from larger conjugates, including 5% S₃-dye. Consequently, removing S₁ and S₂ at some point in the process, before applying the dye conjugates to the fiber, will lead to a 10-fold decrease of washed-out dye and hence to a substantial improvement in washfastness.

This prediction was substantiated when we conjugated a chromophore to the mixture represented in FIG. 10, which has a low content of the small XGO fragments.

The wash-fastness test on a piece of cotton fabric dyed with the resulting XGO-dye conjugate gave a fastness rating of 4-5. Although, due to equipment failure, the result has to be taken as preliminary, this represents a considerable improvement over the unpurified dyes. That the rating was not the highest possible (5) is most likely due to the presence of unconjugated dye molecules.

The binding rate of xyloglucan conjugates can further be increased by partially removing galactosyl residues with beta-galactosidase. As shown in Table 4, binding efficiency of the beta-galactosidase digest is increased relative to undigested XGO-dye conjugates.

Chromophores that do not inherently possess substantivity for cellulose need at least S₃ for good binding to cellulose. However, the binding of chromophores that already have substantivity (e.g. direct dyes) can be enhanced by attaching S₁ or S₂. Symmetrical direct dyes can be furnished with XGO on both ends. Optimum affinity of the conjugate for cellulose can be achieved by choosing the linkage between chromophore and XGO such that their spacing allows both to cooperate in the binding. If the linkage is not of the correct length, not all parts of the conjugate can participate in binding interactions with the cellulose surface, whereas with the ideal linker, both XGO portions and the chromophore line up with the glucosyl residues of the cellulose and can bind cooperatively to them.

While the foregoing description teaches the principles of the present invention, it will be understood that the practice of the invention encompasses all of the usual variations, adaptations, or modifications, as come within the scope of the following claims and their equivalents. Moreover, the invention as disclosed herein, may be suitably practiced in the absence of the specific elements, which are disclosed herein.

All references cited in the present application are incorporated by reference herein to the extent that they are not inconsistent with the present disclosure.

### EXAMPLES

### Example 1

Partial digestion of xyloglucan with endoglucanase and reductive amination with aniline: Tamarind xyloglucan (1.0 g) was dissolved in 100 ml 50 mM acetate buffer (pH 5.0) and treated with 1000 U endo-glucanase (“endo-cellulase” from Megazyme, Cat. No. E-CELTR). After agitating the mixture for 30 min at 20° C., 1.0 M acetic acid was added to bring the pH to 3.85, followed by addition of 1.0 ml aniline. The mixture was stirred for 15 min at 70° C., cooled, treated with 100 mg NaCNBH₃, and stirred for 4 h at 70° C. The solution was dialyzed (MWCO 1000) against 50 mM acetate buffer (pH 5.0) (5×4 l).

### Example 2

Azo coupling of XGO-aniline: The XGO-aniline solution from Example 1 was treated, at 0° C., with 800 μl diazonium salt suspension (prepared from 173 mg sulfanilic acid with 6 M HCl (500 μl) and 2.5 M NaNO₂ (400 μl)), stirred for 18 h at 4° C., and purified by ultrafiltration (MWCO 3000).

### Example 3

Complete digestion of xyloglucan with endoglucanase: Tamarind xyloglucan (2.0 g) was dissolved in 200 ml 50 mM acetate buffer (pH 5.0) and treated with 300 U endo-glucanase (“endo-cellulase” from Megazyme, Cat. No. E-CELTR). After agitating the mixture for 24 h at 50 CC, it was boiled for 5 min, filtered, and lyophilized to give 2.6 g 77% S₁.

### Example 4

Reductive amination of S₁ with aniline and subsequent azo coupling: S₁ (274 mg) was dissolved in 30 ml 50 mM acetate buffer (pH 4.5) and treated with 137 μl aniline. After 30 min at 60° C., the mixture was cooled to 0° C. and 274 μl 10% NaCNBH₃ in buffer was added. The solution was heated at 70° C. for 4 h and subsequently dialyzed (MWCO 1000) against 50 mM acetate buffer (pH 5.0) (3×4 l). The resulting solution (37 ml) was treated, at 0° C., with 150 μl diazonium salt suspension (prepared from 173 mg sulfanilic acid with 500 μl of 6 M HCl and 400 μl of 2.5 M NaNO₂). The mixture was stirred at 4° C. for 18 h, loaded onto a C₁₈ column, and eluted with a MeOH-water gradient (0-50%). The colored (λₘₐₓ=448 nm), carbohydrate-containing, anthrone-positive fractions were pooled and lyophilized to give 117 mg S₁-dye conjugate.

### Example 5

Reductive amination of S₁ with 5-amino-l-naphthol and subsequent azo coupling: S₁ (11.2 mg) was dissolved in 800 μl 50 mM acetate buffer (pH 4.5) and treated with a solution of 13.4 mg 5-amino-l-naphthol in 200 μl acetic acid. After 15 min at 70° C., the mixture was cooled to 0° C., and 10 μl 10% NaCNBH₃ in buffer was added. The solution was heated at 70° C. for 2 h, cooled; filtered, and purified by C₁₈ reverse-phase chromatography (elution with 0-50% gradient water-methanol). The fractions that tested positive for both carbohydrate (anthrone) and arylamine (diazotized sulfanilic acid) were pooled and the resulting solution was treated, at 0° C., with 10 μl diazonium salt suspension (prepared from 173 mg sulfanilic acid with 6 M HCl (500 μl) and 2.5 M NaNO₂ (400 μl)). The mixture was stirred at 4° C. for 18 h, loaded onto a C₁₈ column, and eluted with a MeOH-water gradient (0-50%). The colored (λₘₐₓ=448 nm), carbohydrate-containing (anthrone) fractions were pooled and lyophilized to give 3.2 mg S₁-dye conjugate.

### Example 6

XET-catalyzed coupling of S₁-dye with xyloglucan: Tamarind xyloglucan (100 mg) was dissolved in 6 ml 50 mM acetate buffer (pH 5.0). A solution of 25 mg S₁-dye (see Example 4) in 1 ml 50 mM acetate buffer (pH 5.0) was added, quickly followed by addition of 4.8 ml XET. After 24 h at 24° C., the solution was boiled for 5 min, filtered (0.45 μm), and passed through two consecutive ultrafiltration membranes (MWCO 10,000 and 5000, respectively). The material that passed through the first membrane but not through the second (“5K retentate”) was used to determine the propensity of the S₁-dye conjugate to bind to cotton.

### Example 7

Binding of variously sized XGO-dye molecules to cotton: A 1.0-ml portion of the 5K retentate of XGO-dye was added to 100 mg cotton (3×3 mm squares) and incubated at various temperatures (25, 45, 65, and 85° C.). Aliquots of 200 μl of the supernatant were removed periodically and analyzed by size-exclusion chromatography, using the absorption of light by the dye at 448 nm to determine the amount of each component. The quantity of each different-sized dye peak, which ranged in size (number of S₁-oligosaccharide repeats with a single dye molecule at the reducing end) from S₁-dye to S₇-dye, was determined both before and after exposing the mixture to cotton (results in Table 3).

TABLE 3
Binding of XGO-dye to cotton.

| | | S₁- | S₂- | S₃- | S₄- | S₅- | S₆- | S₇- |
|---|---|---|---|---|---|---|---|---|
| | | dye | dye | dye | dye | dye | dye | dye |
| 25° C. | | | | | | | | |
| 0.5 | h | 11% | 26% | 33% | 34% | 36% | 40% | 35% |
| 2 | h | 9% | 32% | 46% | 47% | 48% | 51% | 52% |
| 4.5 | h | 6% | 34% | 54% | 57% | 58% | 60% | 60% |
| 24 | h | 13% | 36% | 81% | 87% | 89% | 90% | 91% |
| 45° C. | | | | | | | | |
| 0.5 | h | 7% | 26% | 38% | 38% | 40% | 42% | 43% |
| 2 | h | 6% | 31% | 55% | 57% | 58% | 60% | 59% |
| 4.5 | h | 7% | 30% | 66% | 71% | 73% | 76% | 74% |
| 24 | h | 3% | 25% | 86% | 95% | 97% | 97% | 97% |
| 65° C. | | | | | | | | |
| 0.5 | h | 6% | 26% | 44% | 45% | 47% | 47% | 51% |
| 2 | h | 6% | 25% | 61% | 66% | 68% | 72% | 68% |
| 4.5 | h | 4% | 22% | 72% | 81% | 84% | 86% | 87% |
| 24 | h | 4% | 17% | 87% | 97% | 98% | 99% | 99% |
| 85° C. | | | | | | | | |
| 0.5 | h | 4% | 20% | 49% | 53% | 55% | 58% | 57% |
| 2 | h | 3% | 18% | 64% | 76% | 79% | 79% | 82% |
| 4.5 | h | 3% | 15% | 73% | 89% | 92% | 94% | 94% |
| 24 | h | 3% | 12% | 82% | 98% | 99% | 100% | 100% |

### Example 8

Influence of removing galactosyl residues on binding of XGO-dye to cellulose: A 2.0-ml portion of the 5K retentate from Example 5 was mixed with 40 μl 1.0 M acetate buffer (pH 5.6) and then 16 U β-galactosidase was added. The mixture was incubated at 50° C. Half of the mixture (“A”) was removed after 6.5 h, boiled for 5 min, filtered, and analyzed by size-exclusion chromatography. The remainder (“B”) was allowed to react for additional 6.5 h and then analyzed. Both A and B were incubated with 100 mg cotton at 65° C. Aliquots of 200 μl of the supernatant were removed periodically and analyzed by size-exclusion chromatography. The areas of the different XGO-dye components, ranging in size from S₁-dye to S₇-dye were compared to those of the mixture before addition of cotton (see Table 4). Since there was no clear separation between the individual components, the area of peaks with retention times between S₃ and S₇ were summed (see column in Table 4 labled XGO-dye).

TABLE 4
Binding of galactosidase-treated XGO-dye to cotton.

| | | A | B | XGO-dye |
|---|---|---|---|---|
| 0.5 | h | 64% | 72% | 45% |
| 2 | h | 89% | 92% | 65% |
| 4.5 | h | 98% | 98% | 78% |
| 24 | h | 99% | 100% | 93% |

### Example 9

The following protocol relates to the approach where XGO-dye conjugates are formed first and then followed by endoglucanase digestion. Azo-xyloglucan (0.5 g, Megazyme Cat. No. S-AZXG) was dissolved in 40 ml hot water (40 ml). Sodium acetate buffer (2.1 ml of a 1 M solution, pH 5) was added, followed by endoglucanase (20 μl of a 900 U/ml suspension, Megazyme Cat. No. E-CELTR). The mixture was stirred at 25° C. for 2 h, after which time the reaction was terminated by adding sodium hydroxide (0.5 M) until pH 8 was reached. The enzyme was destroyed by heating the mixture to boiling for 5 min. The mixture was filtered through Celite, placed into a 100 ml beaker, and heated. When the solution reached a temperature of 90-95° C., a piece of mercerized cotton (1 60×75 mm, 1.32 g), folded 3 times, was immersed into it and dyed at 90-95° C. for 30 min. Subsequently, the fabric was rinsed with warm water and washed by soaking in 600 ml water at 80° C. for 20 min. After drying, the fabric was dyed a medium blue shade.

### Example 10

Tamarind xyloglucan (100 mg) was dissolved in sodium acetate buffer (10 ml of a 20 mM solution, pH 5). The mixture was treated with endoglucanase (1 μl of a 900 U/ml suspension, Megazyme Cat. No. E-CELTR). After 30 min, the pH was adjusted to 8, and the solution was heated to boiling for 5 min. After filtration through Celite, the solution was concentrated by ultrafiltration (10 kDa MWCO) to a volume of 4 ml. To the partially depolymerized solution of xyloglucan was added a solution of 20 mg Reactive Blue 4 in 330 μl water. Sodium sulfate (440 mg) was added in portions during 30 min while stirring the mixture at 25° C. Subsequently, the solution was brought to pH 12 by addition of 0.5 M trisodium phosphate and stirred for 15 min at 25° C. and for 30 min at 55° C. After filtration through Whatman type 1 filter paper, the solution was desalted by ultrafiltration (10 kDa MWCO). After adjusting the pH of the solution to 5, the product was further depolymerized by endoglucanase (4 μl suspension 900 U/ml) for 2 h at 25° C. After deactivation of the enzyme in the usual way, the solution was used to dye a piece of cotton fabric (55×110 mm, 0.7 g), and a light blue shade was obtained.

### Example 11

Synthesis of (4-aminomethylphenyl)amino-S₁ (S₁-4-ABA): To a solution of 1.0 g S₁ in 10 ml 50 mM sodium acetate buffer, pH 3, was added 81 μl 4-aminobenzylamine. The solution was brought to pH 3.68 by addition of glacial acetic acid, and 45 mg sodium cyanoborohydride was added. The mixture was heated to 70° C. for 3 h. After cooling to 25° C., the mixture was filtered, and the product was isolated by gel filtration (Sephadex® G-25) and lyophilization to give 855 mg S₁-4-ABA.

Synthesis of (4-aminomethylphenyl)amino-XGO (XGO-4-ABA): To a solution of 686 mg XGO in 7 ml 50 mM sodium acetate buffer, pH 3, was added 9.3 μl 4-aminobenzylamine. The solution was brought to pH 3.98 by addition of glacial acetic acid, and 9.8 mg sodium cyanoborohydride was added. The mixture was heated to 70° C. for 4 h. After cooling to 25° C., the mixture was filtered, and the product was isolated by gel filtration (Sephadex® G-25) and lyophilization to give 465 mg XGO-4-ABA.

To a rapidly stirred suspension of 17.8 mg cyanuric chloride in 100 μl water/acetone (Thurston et al., 1951) was added, at 0° C., a solution of 12 mg S₁-4-ABA in 150 μl water, followed by 10 μl 2M sodium carbonate. The mixture was stirred at 0° C. for 2 h, after which time it was filtered through Celite and treated with 50 μl octylamine. The mixture was then heated with stirring to 40° C. for 1 h and to 110° C. for 2 h. After filtration through Celite and separation of the liquid octylamine from the filtrate, the aqueous phase was washed with chloroform and freeze-dried to give 3 mg of product.

MALDI-TOF mass spectrometry confirmed the presence of 6a (X═C₈H₁₇NH, see FIG. 12) as major product and 3a (X═C₈H₁₇NH, XGO═S₁) as minor product (ratio ˜4:1).

To a rapidly stirred suspension of 17.8 mg cyanuric chloride in 100 μl water/acetone (Thurston et al., 1951) was added, at 0° C., a solution of 12 mg S₁-4-ABA in 150 μl water. The mixture was stirred at 0° C. for 1 h, after which time it was filtered through Celite and treated with 60 mg dodecylamine. The mixture was then heated with stirring to 40° C. for 30 min and to 110° C. for 2 h. After filtration through Celite and separation of the liquid dodecylamine from the filtrate, the aqueous phase was washed with chloroform and freeze-dried to give 6 mg of product.

MALDI-TOF mass spectrometry confirmed the presence of 3b (X═C₁₂H₂₅NH, XGO═S₁, see FIG. 12) as major product.

1. A method of making a xyloglucan conjugate comprising the steps of:
(a) preparing xyloglucan fragments from xyloglucan polymers; and
(b) attaching one or more functional groups to the reducing end and/or side chains of the xyloglucan fragments whereby a xyloglucan conjugate useful for binding to cellulosic material is produced.

2. The method of claim 1 wherein said xyloglucan fragments are prepared by enzymatic digestion.

3. The method of claim 2 wherein said enzymatic digestion is carried out by employing β-1,4-endoglucanase.

4. The method of claim 1 wherein said xyloglucan fragments are a mixture of oligosaccharides ranging in size up to five hundred glycosyl residues.

5. The method of claim 4 wherein said xyloglucan fragments are a mixture of oligosaccharides ranging in size up to three hundred glycosyl residues.

6. The method of claim 1 wherein said xyloglucan conjugate comprises more than one type of functional group per xyloglucan fragment.

7. The method of claim 1 wherein said xyloglucan fragments consist of up to 60 randomly ordered hepta-, octa-, and nonasaccharide subunits, each of which has a β-1,4-tetraglucoside backbone.

8. The method of claim 1 wherein said functional group is a dye molecule.

9. The method of claim 8 wherein said dye molecule is an azo dye.

10. The method of claim 1 wherein said functional group is selected from the group of compounds useful as a fabric softener, antimicrobial agent, water repellant, oil repellant or a firming agent.

11. The method of claim 1 wherein said functional group is an aromatic amine.

12. The method of claim 11 wherein said functional group is attached in a 2-step process comprising (i) attaching an aromatic amine and (ii) performing an azo coupling on the resulting carbohydrate conjugate.

13. The method of claim 12 wherein said aromatic amine is attached by reductive amination.

14. The method of claim 12 wherein said aromatic amine is attached by electrolytic oxidation followed by amide bond formation.

15. The method of claim 12 wherein said aromatic amine is attached by carbon-carbon bond formation between xyloglucan fragments and a heterocyclic compound.

16. The method of claim 15 wherein said heterocyclic compound is a pyrazolinone derivative.

17. A xyloglucan conjugate capable of binding to cellulosic material.

18. The xyloglucan conjugate of claim 17 comprising a dye molecule.

19. The xyloglucan conjugate of claim 18 wherein said dye is an azo dye.

20. The xyloglucan conjugate of claim 17 comprising a functional group useful as a fabric softener, fluorescent brightening agent, lubricant, antimicrobial agent, water repellent, oil repellent, or a firming agent.

21. The xyloglucan conjugate of claim 17 wherein the cellulosic material is cotton.

22. A method of attaching a functional group to cellulosic material comprising the steps of:
(a) preparing xyloglucan fragments from xyloglucan polymers by hydrolysis;
(b) attaching one or more functional groups to the reducing end and/or side chains of the xyloglucan fragments to produce a xyloglucan conjugate; and
(c) treating a cellulosic material with the xyloglucan conjugate whereby the cellulosic material containing the functional group is produced.

23. The method of claim 22 wherein said functional group is a dye molecule.

24. The method of claim 22 wherein the xyloglucan conjugate comprises more than one type of functional group per xyloglucan fragment.

25. The method of claim 22 wherein said functional group is selected from the group of compounds useful as a lubricant, fluorescent brightening agent, fabric softener, antimicrobial agent, water repellant, oil repellant or a firming agent.

26. The molecule of claim 23 wherein said dye molecule is an azo dye.

27. The method of claim 22 wherein said cellulosic material is cotton.

28. The method of claim 22 wherein said hydrolysis step is carried out by using an enzyme selected from the group consisting of β-galactosidase, β-1,4-endoglucanase, and xyloglucan endotransglycosidase (XET).

29. The method of claim 28 wherein said enzyme is β-1,4-endoglucanase.

30. The method of claim 22 wherein said xyloglucan fragments consist of up to 60 randomly ordered hepta-, octa-, and nonasaccharide subunits, each of which has a β-1,4-tetraglucoside backbone.

31. The method of claim 22 wherein said xyloglucan conjugates are treated with beta-galactosidase.

32. The method of claim 22 wherein said xyloglucan fragments are purified by ultrafiltration.

33. The method of claim 22 wherein said xyloglucan conjugates are purified by ultrafiltration.

34. A method of attaching a functional group to cellulosic material comprising the steps of:
(a) attaching one or more functional groups to the side chains of xyloglucan polymers to form modified xyloglucan polymers;
(b) preparing a xyloglucan conjugate from the modified xyloglucan polymers of (a) by hydolysis; and
(c) treating a cellulosic material with the xyloglucan conjugate whereby the cellulosic material containing the functional group is produced.

35. The method of claim 34 wherein said functional group is a dye molecule.

36. The method of claim 34 wherein the xyloglucan conjugate comprises more than one type of functional group per xyloglucan fragment.

37. The method of claim 34 wherein said functional group is selected from the group of compounds useful as a lubricant, fluorescent brightening agent, fabric softener, antimicrobial agent, water repellant, oil repellant or a firming agent.

38. The method of claim 35 wherein said dye molecule is an azo dye.

39. The method of claim 34 wherein said cellulosic material is cotton.

40. The method of claim 34 wherein said hydrolysis step is carried out by enzymatic digestion.

41. The method of claim 40 wherein said enzymatic digestion is carried out by employing endoglucanase.

42. The method of claim 34 wherein said xyloglucan conjugate is purified by ultrafiltration.
